(19) United States
(12) Patent Application Publication
(10) Pub. No.: US 2010/0173016 A1
(43) Pub. Date: Jul. 8, 2010

(54) COMPOSITIONS AND METHODS FOR THE ABSORPTION, CHELATION, AND ELIMINATION OF TRACE METALS

(76) Inventor: Paul J. Reynolds, Seattle, WA (US)

Correspondence Address: Nancy Lord, Ltd, 1970 N. LESLIE RD., NO. 220, PAHRUMP, NV, 89060, US

(21) Appl. No.: 12/350,856

(22) Filed: Jan. 8, 2009

Related U.S. Application Data
(62) Division of application No. 12/350,538, filed on Jan. 8, 2009.

(51) Int. Cl. A61K 33/06

(52) U.S. Cl. 424/684

(57) ABSTRACT

This invention relates to compositions and methods effective for the absorption, chelation and elimination of trace elements, in particular tin, mercury, aluminium, cadmium, lead, and other metals, metaloids and lanthinides. It is comprised of dihydroquercetin and zeolite, with or without humic and fulvic acids. The combination acts as a powerful chelator and eliminator of toxic trace elements and enhances their absorption from the intestine, blood, and organs, and may be administered for the absorption, chelation and elimination of toxic or potentially toxic trace elements including metals and metalloids, and lanthinides.

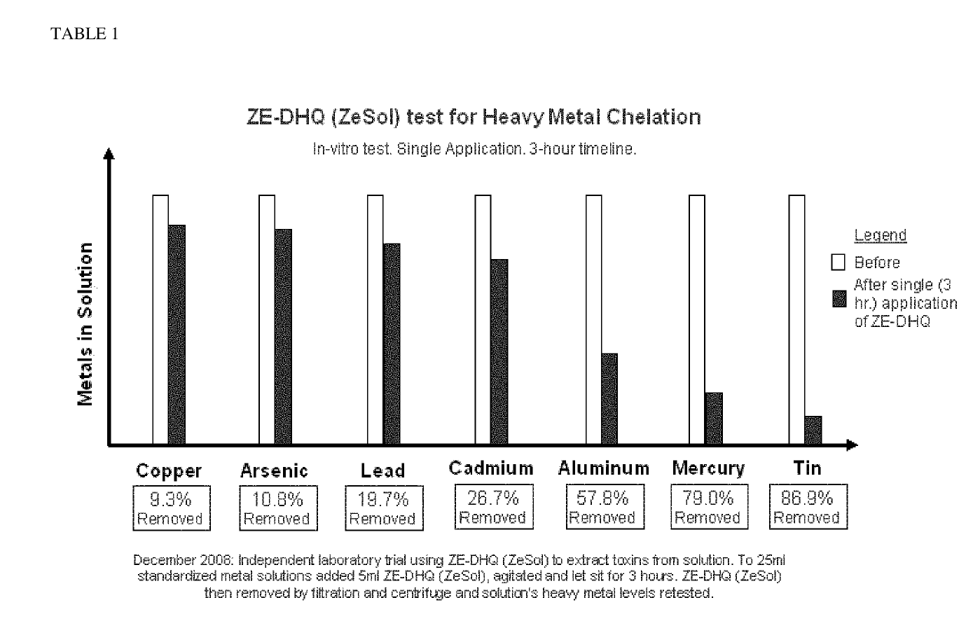

### FIELD OF INVENTIONS

This invention relates to compositions and methods effective for the chelation, absorption and elimination of trace elements comprised of dihydroquercetin and zeolite, with or without humic and fulvic acids.

### BACKGROUND OF INVENTION

Trace Metal Toxicology

The dangers of the accumulation of trace elements are well established, especially transition metals, poor metals, particularly lead, and metalloids such as arsenic. Chronic low-level intakes of heavy metals have damaging effects on human beings and other animals, since there is no good mechanism for their elimination. Metals such as lead, mercury, cadmium and copper are cumulative poisons. These metals cause environmental hazards and are reported to be exceptionally toxic. Vegetables take up metals by absorbing them from contaminated soils, as well as from deposits on parts of the vegetables exposed to the air from polluted environments. Islam, E u, et al, 8(1) J Zhejiang Univ Sci B. 1-13 (2007).

Geoscientists working with medical researchers and public health scientists have made important contributions to understanding novel exposure pathways and causes of a wide range of environmental health problems such as exposure to toxic levels of trace essential and non-essential elements such as arsenic and mercury. Finkelman R B, Centeno J A, Selinus 0,116 Trans Am Clin Climatol Assoc 155-65 (2005). Even essential trace elements that are required for mammalian homeostasis, such as manganese, iron, copper, and zinc, have adverse effects on animals that have accumulated them in excessive quantities. The average contents of lead, cadmium, zinc and manganese are higher in the semen than in the follicular fluid in the non-professionally exposed infertile couples. Zha, S W, 14(6) Zhonghua Nan Ke Xue 494-7 (2008). While generally referred to as “heavy metals,” several of the elements known to accumulate in mammalian systems are really semi-metals or metalloids and have properties somewhere between the two categories of metals and non-metals or exhibit some of the qualities of both. The semi-metals or metalloids (meaning metal-like) include silicon (Si), germanium (Ge), arsenic (As), antimony (Sb), and tellurium (Te). Some chemists also include boron (B), aluminum (Al), polonium (Po), and astatine (At) in the semi-metals.

The accumulation or release of trace elements in wetland sediments is controlled largely by their geochemistry, with reducing and oxidation (redox) behavior playing a particularly important role. In general arsenic (As), molybdenum (Mo), and vanadium (V) concentrations in the sediment were highest under more reducing conditions and lowest under more oxidizing conditions. Most of the accumulated Mo (73%) became water soluble on drying of samples. This has important implications for systems undergoing changes in redox status; for instance, if these wetland sediments are dried, potentially large amounts of Mo may be solubilized. Fox, P M, and Donner, H E, 32 J. Environ. Qual. 2428-2435 (2003).

Selenium (Se) is an essential trace element and potent anti-oxidant for humans and other animals, and there is mounting evidence for the efficacy of certain forms of selenium as cancer chemopreventive compounds. However, over the years, numerous elements such as As, Cu, Zn, Cd, Hg, Sn, Pb, Ni, Co, Sb, Bi, Ag, Au, and Mo have been found to affect the anti-carcinogenic activity of selenium. The interaction between selenium and arsenic, a metalloid, has been one of the most extensively studied. The proposed mechanisms of this interaction include the increase of biliary excretion and direct interaction/precipitation of selenium and arsenic, and their effects on zinc finger protein function, cellular signaling and methylation pathways. Zeng H, Uthus E O, Combs G F Jr., 99(6) J Inorg Biochem 1269-74 (2005).

At higher concentrations, Selenium (Se) compounds, can be either cytotoxic or possibly carcinogenic. The cytotoxicity of Se is suggested to be associated with oxidative stress. Accordingly, sodium selenite, an inorganic Se compound, was reported to induce DNA damage, particularly DNA strand breaks and base damage. Letavayová L, Vlcková V, Brozmanová J, 227(1-2) Toxicology 227(1-2):1-14 (2006).

Alzheimer's disease (AD) is a progressive neurodegenerative disorder and the most common cause of dementia, affecting millions of men and women worldwide. It is characterized by the accumulation of extracellular amyloid-beta (A beta) plaques and neurofibrillary tangles inside neurons and dystrophic neurons. Several risk factors are associated with the early onset and progression of the disease. Although the initiating molecular events are not entirely known, in recent years it has become evident that environmental and/or nutritional factors may play a causal, disruptive, and/or protective role in the development of AD. While a direct causal role for aluminum or other transition metals (copper, zinc, iron) in AD has not yet been definitively demonstrated, epidemiological evidence suggests that elevated levels of these metals in the brain may be linked to the development or progression of AD. Shcherbatykh I, Carpenter D O, 11(2) J Alzheimers Dis 191-205 (2007).

Mercury (Hg), still to this day used in some dental amalgam, has been shown to accumulate and become toxic to humans and animals throughout the body compartments, especially the liver and kidney. Santarelli L, et al 29(3 Suppl) G Ital Med Lay Ergon. 534-6 (2007). This Italian research group also investigated the effects of other essential metals' homeostasis, in particular that of copper, zinc, manganese and iron in Hg chronic intoxication. The effects of L-arginine, that induces beneficial influence on immunologic functions on mice intoxicated with Hg, was also studied as a detoxifier of Hg with good restoration to normal homoeostatic conditions. Another research group has proposed, based on recent studies, a novel paradigm for the pathogenesis of viral dilated cardiac myopathy that incorporates trace element imbalance between selenium and mercury and its interactions with the cellular physiology of viral-induced cardiomyocyte dysfunction. Cooper L T, Rader V, Ralston N V, 13(4) Congest Heart Fail 13(4):193-9 (2007).

It appears that Mercury (Hg) represents the most toxic metal at environmentally relevant concentrations on human peripheral mononuclear cells. Among environmental contaminants recognized for their toxicity and global distribution, the so-called heavy metals are elements known to exert serious ecological consequences. Published experiments on the immunotoxic effects of metals such as methylmercury (MeHg), cadmium (Cd), and lead (Pb) were often conducted at concentrations higher than those present in the environment or those in human blood. Results showed an increase of intracellular thiols in lymphocytes and in monocytes at all the concentrations of metals tested. A decrease in the level of metallothionein (MT) was seen in monocytes in presence of Hg at concentration of 120 mug/L and higher. For lymphocytes, a significant increase of MT in groups containing the lower concentrations of Cd and Hg was noted. The effects of Hg exposure were greater on lymphocytes and NK cells than on monocytes. Fortier, M, et al, 71(19) J Toxicol Environ Health A 1327-37 (2008).

Arsenic, a carcinogenic trace element, threatens not only the health of millions of humans and other living organisms, but also global sustainability. In 2000, the world cumulative industrial age anthropogenic arsenic production was 4.53 million tons. The world-wide coal and petroleum industries accounted for 46% of global annual gross arsenic production, and their overall contribution to industrial-age gross arsenic production was 27% in 2000. The development of substitute materials for arsenic applications in the agricultural and forestry industries and controls of arsenic emissions from the coal industry may be possible strategies to significantly decrease arsenic pollution sources and dissipation rates into the environment. Han, F X, et al, 90(9) Naturwissenschaften 395-401 (2003).

Despite the well-known toxicity of aluminum in chronic renal failure, a solid database on its biokinetics has been difficult to establish. Aluminum absorption, distribution, speciation and excretion in six healthy volunteers and in two patients with chronic renal failure were investigated following administration of a single oral or i.v. dose of (26)Al. Serial samples of blood and urine were taken. In a speciation study, the time dependence of the binding of (26)Al to low-molecular weight molecules in serum was investigated. Differences between healthy volunteers and patients with chronic renal failure were deduced. Steinhausen, C, et al, 42(3) Food Chem Toxicol 363-71 (2004).

Manganese (Mn), an essential trace metal element required for ubiquitous enzymatic reactions has been shown to promote neurotoxicity with overexposure. While the mechanism of Mn toxicity is not well established, several studies indicate that oxidative stress and mitochondria play major roles in the Mn-induced neurodegenerative processes that lead to dysfunction in the basal ganglia. Researchers confirmed the oxidative hypothesis of Mn cytotoxicity with coexposure of MnCl(2) and antioxidant agents (N-acetylcysteine [NAC] or Trolox). A significant decrease in Mn cytotoxicity was observed in co-exposed cells confirming that (1) oxidative stress plays a critical role in the mechanism of Mn toxicity, and (2) antioxidants may offer a useful therapeutic modality. Marrielha, D S, et al Brain Res. (2008). Other groups had previously concluded that oxidative stress generated through mitochondrial perturbation might be a key event in the demise of central nervous system cells affected by manganese. Studies with primary astrocyte cultures have revealed that they are a critical component in the battery of defenses against manganese induced neurotoxicity. Dobson A W, Erikson K M, Aschner M, 1012 Ann NY Acad Sci 115-28 (2004). Uranium and lead have been shown to be distributed to osteoblastic cells and accumulate there.

Milgram S, et al 252 (1-3) Toxicology 26-32. Epub 2008 Jul 31. These researchers showed that Uranium (U) and lead (Pb) are accumulated and fixed for long periods in bone, impairing remodeling processes. Their toxicity to osteoblasts, the cells responsible for bone formation, had been poorly documented, though it was previously shown that cytotoxicity and phenotypic effects of both metals on osteoblasts are highly influenced by metal speciation. Cellular accumulation of U and Pb in cultured and primary osteoblastic cells was assessed by trace element analysis by electron microscopy and the internalization of both metals was shown to be correlated to cytotoxicity and population growth recovery after exposure.

Trivalent chromium [Cr(III)] is recognized as an essential nutrient, and is widely used as a nutritional supplement for humans and animals both in weight control and modulation of insulin sensitivity. But recent reports of the induction of genetic damage in cultured cells exposed to Cr(III) compounds in vitro prompted a review of the literature since 1990 on genotoxic effects of Cr(III) compounds to determine whether recent findings provide a sufficient weight of evidence to modify the conclusions about the safety of this dietary supplement reached in the several comprehensive 1990-2004 reviews. The in vitro data show that Cr(III) has the potential to react with DNA and to cause DNA damage in cell culture systems, but under normal circumstances, restricted access of Cr(III) to cells in vivo limits or prevents genotoxicity in biological systems. While, the available in vivo evidence suggests that genotoxic effects are very unlikely to occur in humans or animals exposed to nutritional or to moderate recommended supplemental levels of Cr(III), excessive intake of Cr(III) supplements did not appear to be warranted at this time. Eastmond D A, Macgregor J T, Slesinski R S, 38(3) Crit Rev Toxicol 173-90 (2008).

Vanadium, a trace element useful as a supplement for modulation of insulin sensitivity, Srivastava A K, Mehdi M Z, 22(1) Diabet Med 2-13 (2005), is known to interfere with a wide variety of enzymes in the form of vanadate, including Ca2+ ATPase and Na+/+ ATPase. Vanadate is excreted mainly via the kidney and impaired renal function and its consequent impaired vanadate excretion leads to its accumulation in blood. Researchers studied the effect of vanadate on eryptosis, the suicidal death of erythrocytes. The researchers concluded that vanadate induces eryptosis at least partially through increase of cytosolic Ca2+ concentration, an effect presumably contributing to the development of anemia in chronic renal failure. Foller, M, et al, 31(2) Kidney Blood Press Res 87-93 (2008).

Lanthanides such as yterrbium (Yb) because of their diversified physical and chemical effects, have been widely used in a number of fields. As a result, more and more lanthanides are entering the environment and eventually accumulating in the human body. Studies indicated that the impact of lanthanides on brain function cannot be neglected, leading researchers to study the status of micronutritional elements in rats after prenatal and long-term exposure to lanthanide, yterrbium (Yb) and its trace elements distribution in brain and organic tissues of offspring rats after prenatal and long-term exposure to Yb. The accumulation of Yb in the brain, liver, and femur was observed; and moreover, the levels of Fe, Cu, Mn, Zn, Ca, and Mg in the brain and organic tissues of offspring rats were also altered after Yb exposure, possibly inducing adverse effects on normal physiological functions of the brain and other organs. Feng, L, et al, 117 (1-3(Biol Trace Elem Res 89-104 (2007).

Previous research has suggested that certain trace elements known informally as “heavy metals” may be ototoxic in humans, and further, that a reversal of this toxicity may occur when the trace element selenium is present, through formation of metals selenide complexes. A case control study of the relationship between hearing thresholds and blood concentrations of four elements (selenium, lead, manganese, and arsenic) was performed in factory workers in Taiwan. In our regression models, age, lead and selenium concentrations (logarithmic transformed) were associated significantly with hearing thresholds. In addition, the Taiwanese researchers found that the selenium, a potent anti-oxidant, was inversely associated with hearing thresholds, and may be an antagonist to lead ototoxicty and a dose-response relationship between blood lead and hearing thresholds was found. Chuang, H Y, 387(1-3) Sci Total Environ 79-85 (2007).

Cadmium (Cd2+) is an environmental contaminant showing a variety of adverse effects. Given the current rate of release into the environment, the amount of Cd2+ present in the human body and the incidence of Cd2+-related diseases are expected to increase. In a study of the mechanism of Cd2+-induced ototoxicity, cell viability, reactive oxygen species (ROS), mitochondrial membrane potential (MMP), cytochrome c (cyt c), phosphorylated extracellular signal-regulated protein kinase (p-ERK), caspases, morphologic change, and functional changes in HEI-OC1 cells, rat cochlear explants, and mouse cochlea after Cd2+ exposure were measured by flow cytometry, immunohistochemical staining, Western blot analysis, and auditory brainstem response (ABR) recording. ROS generation may be the cause of the toxicity, and application of antioxidants can prevent the toxic effect.

Fractional cadmium uptake is of fundamental importance for internal dose, related individual susceptibility to cadmium, induced renal damage and eventually bone disease. Diet composition with regard to macronutrients has some effects on cadmium bioavailability. Major determinants of intestinal cadmium uptake are however diet composition with regard to crude fibers and trace elements, especially iron. Deficiencies may increase intestinal cadmium uptake 5-8 times. Ultimate risk management would be not to raise crops on cadmium polluted soil. Provisionally, assurance of optimal trace element status in persons exposed to cadmium is essential for risk reduction. Andersen O, Nielsen J B, Nordberg G F, 17(5) Biometals 543-7 (2004).

Mineral particles in occupational exposure and ambient air particles may cause adverse health effects in humans. Stone quarry particles were studied to induce release of the proinflammatory cytokines interleukin-6 (IL-6) and interleukin-8 (IL-8) from human epithelial lung cells (A549). Particles, containing minerals such as quartz, amphibole, chlorite, and epidote, induced a marked increase in IL-6 and IL-8 release. Particles composed mainly of plagioclase were much less effective. The most potent particle samples exhibited a relatively high content of transition metals such as iron. Hetland, R B, et al, 60(1) J Toxicol Environ Health A 47-65 (2000). Crystalline silica has been classified as a human carcinogen, but there is still considerable controversy regarding its fibrogenic and carcinogenic potential. In a present study investigating the genotoxic potential of bentonite particles (diameter <10 microm) with an a-quartz content of up to 6% and different chemical modifications (alkaline, acidic, organic) human lung fibroblasts (IMR90) were incubated for 36 h, 48 h, or 72 h with bentonite particles in concentrations ranging from 1 to 15 microg/cm2. Genotoxicity and the generation of reactive oxygen species (ROS) caused by bentonite particles via Fenton-like mechanisms was measured. Altogether, it was concluded that the genotoxic potential of bentonite particles is generally low but can be altered by the content of quartz and available transition metals. Geh, S, et al, 18(6) Inhal Toxicol 18(6):405-12 (2006).

The transition metals such as iron, vanadium, and nickel, have been implicated in their role in the production of free radicals and their metabolites, also called reactive oxygen species (ROS), that have been implicated in the pathogenesis of many diseases. Because of its continuous exposure to toxic pollutants in the ambient air, such as cigarette smoke, air pollution, and mineral dusts, the lung is very vulnerable to ROS-induced injury. van Klaveren R J, Nemery B, 5(2) Curr Opin Pulm Med 118-23 (1999).

Copper and other transition metals are involved with lipid hydroperoxides in the oxidation of human low density lipoproteins (LDL) that is induced by Cu++ or azoperoxyl radicals. Analysis by high-performance liquid chromatography of lipids extracted from Cu+(+)- or azoperoxyl radical-modified lipoproteins indicated that the major lipid oxidation products were derived from linoleate and that Ebselen-glutathione addition reduced linoleate hydroperoxides. The initiation of Cu+(+)-dependent oxidation absolutely requires the presence of trace amounts of lipid hydroperoxides in the lipoprotein and that hydroperoxides play a major role in the propagation reactions even in the absence of added transition metals. Thomas C E, Jackson R L, 256(3) J Pharmacol Exp Ther 1182-8 (1991).

Organotin compounds, mainly methyltin and dimethyltin (DMT) at concentrations ranging respectively from 0.5 to 257 ng Sn/L and from 0.5 to 6.5 ng Sn/L, were detected in samples from ten of the twenty-two houses on distribution lines where PVC pipe/tubing had been recently installed. Saidiki, A I, et al, 32(12) Chemosphere 2389-98 (1996). Dimethyltin is one of several organotins that are detected in domestic water supplies due to their use as plastic stabilizers for polyvinyl chloride (PVC) and chlorinated PVC (CPVC) products. A limited number of in vitro and in vivo studies suggest that DMT may produce developmental neurotoxicity. Researchers initiated studies to evaluate long-term neurobehavioral changes in offspring following perinatal exposure. DMT toxicity was expressed as depressed maternal weight gain (74 ppm), and in the offspring, decreased brain weight (3, 74 ppm), decreased apoptosis (all concentrations), mild vacuolation in adult offspring (all concentrations), and slower learning in the water maze (15 ppm) due to altered spatial search patterns. In a second study, DMT exposure (same concentrations) occurred from gestational day 6 to weaning. Male and female offspring were also tested and developmental neurotoxicity may be produced in offspring following gestational exposure to DMT in drinking water. Ehman, K D et al, 29(6) Neurotoxicol Teratol 622-33 (2007).

Need for Effective Detoxification

In spite of the known dangers of accumulated trace elements within man and other mammalian bodies, there are no proven means to effectively remove these trace elements. Of the clinical detoxification studies that exist, the majority are observational studies on a detoxification program promoted by the Church of Scientology and the controversial Narconon Drug Rehabilitation Program, received attention after it was used to treat rescue workers exposed to multiple toxins after the World Trade Centre towers disaster. This program involves the use of high dose niacin along with other vitamins, minerals and polyunsaturated oils in conjunction with physical exercise and extensive sweating induced by sauna. A number of case reports, cohort studies, and nonrandomised, controlled trials suggest that this program can reduce the body burden of polychlorinated biphenyls (PCBs), and polybrominated biphenyls (PBBs), dioxins, and various drugs and pesticides, but not trace elements and has not been confirmed by more systematic and rigorously controlled trials. Cohen, Marc 36(12) American Family Physician 1009-10 (2007).

Dihydroquercetin

Dihydroquercetin is a bioflavonoid. Flavonoid natural products display a wide range of biochemical and pharmacological properties, with one of the most thoroughly characterized effects being chemopreventive activity, measured by the chemopreventive index as a marker for the screening of potential agents. Dihydroquercetin, also known as taxifolin, exhibits high detoxification ability but lower cytotoxicity in cells, representative of a high chemopreventive index. It has been shown to modulate the expression of several genes, including those coding for detoxification enzymes, cell cycle regulatory proteins, growth factors, and DNA repair proteins. A recent microarray results show that the phase II detoxification enzymes, NQO1 and GSTM1, are upregulated, while the phase I detoxification enzyme, CYP2E1, is down regulated in the presence of taxifolin. Lee, S B, et al, 30(6) Biol Pharm Bull 1074-1079 (2007). Measurement of cytotoxicity to human lung embryonic fibroblasts (TIG-1) and umbilical vein endothelial (HUVE) cells, was examined and

Dihydroquercetin shown to be the least cytotoxic of the ten (10) tested flavonoids, with a 50% lethal concentration of >300 micromoles in TIG-1 and >200 micromoles in HUVE cells. Matsuo, M, et al, 28(2) Biol Pharm Bull 253-9 (2005).

Dihydroquercetin is a powerful antioxidant and is one substance in foods that significantly decreases the adverse effects of radical oxygen species, and reactive nitrogen species. Valko, M et al, 39(1) Int J Biochem Cell Biol. 44-84 (2007); Kostyuk V A, Potapovich A I, 355(1) Arch Biochem Biophys 43-8 (1998); Soliman K F & Mazzio E A, 218(4) Proc Soc Exp Biol Med 390-7 (1998). It was discovered as an essential molecular intermediate in flavonoid biosynthetic pathways responsible for quercetin, oligo proanthocyanidins (OPC), and catechin formation and its superiority as an anti-oxidant has been demonstrated repeatedly. Tiukavkina N A, Rulenko I A, Kolesnik I A, 6 Vopr Pitan. 12-5 (1997); Teselkin, I O, et al, 41(3) Biofizika. 620-4 (1996).

Dihydroquercetin protects cell membranes from free radical damage, improves the activity of capillaries and supports the recovery of blood microcirculation throughout the body. Oxidative stress is reduced at the cellular level, as shown, for instance in cultured renal cells. Areias, F M, et al, 62(1) Biochem Pharmacol. 111-8 (2001).

Zeolite

The classical definition of a zeolite is a crystalline, porous aluminosilicate. However, some relatively recent discoveries of materials virtually identical to the classical zeolite, but consisting of oxide structures with elements other than silicon and aluminum have stretched the definition. Most researchers now include virtually all types of porous oxide structures that have well-defined pore structures due to a high degree of crystallinity in their definition of a zeolite.

In these crystalline materials called zeolites, the metal atoms (classically, silicon or aluminum) are surrounded by four oxygen anions to form an approximate tetrahedron consisting a center and oxygen anions at the four apexes. The tetrahedral metals are called T-atoms for short, and these tetrahedra then stack in beautiful, regular arrays such that channels form. The possible ways for the stacking to occur is virtually limitless, and hundreds of unique structures are known.

The zeolitic channels (or pores) are microscopically small, and in fact, have molecular size dimensions such that they are often termed “molecular sieves”. The size and shape of the channels have extraordinary effects on the properties of these materials for adsorption processes, and this property leads to their use in separation processes. Molecules can be separated via shape and size effects related to their possible orientation in the pore, or by differences in strength of adsorption.

Since silicon typically exits in a 4+ oxidation state, the silicon-oxygen tetrahedra are electrically neutral. However, in zeolites, aluminum typically exists in the 3+ oxidation state so that aluminum-oxygen tetrahedra form centers that are electrically deficient one electron. Thus, zeolite frameworks are typically anionic, and charge compensating cations populate the pores to maintain electrical neutrality. These cations can participate in ion-exchange processes, and this yields some important properties for zeolites. When charge compensating cations are “soft” cations such as sodium, zeolites are excellent water softeners because they can pick up the “hard” magnesium and calcium cations in water leaving behind the soft cations. When the zeolitic cations are protons, the zeolite becomes a strong solid acid. Such solid acids form the foundations of zeolite catalysis applications including the important fluidized bed cat-cracking refinery process. Other types of reactive metal cations can also populate the pores to form catalytic materials with unique properties. Thus, zeolites are also commonly used in catalytic operations and catalysis with zeolites is often called “shape-selective catalysis”.

Humic Acid

Humic acid, defined as the portion of soil humus that is soluble in alkaline solution, but insoluble in acid solution, is the form of organic matter that often is added to the soil to increase fertility. Humic acids are found in rotting vegetable matter and can be detected in the black slime of an ordinary compost pit in a home garden. It also is found in the brown organic matter of a variety of soils, as well as in peats, manure, lignite, leonardite, brown coals, and the Menefee Humate™. Humic acids do not have a single unique structure, but are a mixture of intermediate chemical products resulting from the decomposition and conversion of lignin and other plant materials to hard coal. Humic acids apparently are formed by the bacterial and chemical degradation of plant tissue, but in soils it also may be formed by certain secondary processes such as polymerization of polyphenols leached by rain from surface leaf litter, and condensation of phenols, quinones, and proteins that are provided by the action of soil micro-organisms and small animals on soil carbohydrates. As a result, humic acid is best characterized in terms of its origin and soil environment, rather than in rigid terms of chemical composition or chemical properties.

Chemical studies of the composition of humates such have revealed that they are mainly composed of the mixed salts of acid radicals found in soil humus, a product of the decay of organic matter that contains both humic and nonhumic material. Such acid radicals are collectively termed “humic acids,” having individual factions named humin, humic acid, ulmic acid and fulvic acid. The exact structure of the humic acids are unknown. However, humic acids appear to be associations of molecules forming aggregates of elongated bundles of fibers at low pH, and open flexible structures perforated by voids at high pH. These voids, of varying dimensions, trap organic or inorganic particles of appropriate electronic charge.

The humic acids have a large cation exchange capacity and hold multivalent metallic elements, such as micronutrient elements, very strongly. The molecular weight of the humic acids range from 800 to 500,000, with the average molecular weight ranging from about 5,000 to about 50,000. The cation exchange capacity of the humic acids varies from about 200 to about 600 meq CaCO.sub.2 per 100 grams at pH 7, depending upon the origin of the extracted acids. Humic acids are polyelectrolytes and are believed to form complexes with clay particles thus enabling humic acids to bind multivalent elements with great tenacity. When the cation exchange sites on the humic acid molecule are filled predominantly with hydrogen ions, the material, considered to be an acid, is insoluble in water. However, when the predominant cations at the exchange sites are other than hydrogen, the material is called a “humate.” Humates of monovalent alkali metals or ammonia are soluble in water, but the humates of most multivalent metals are insoluble.

It has been shown in studies done on poultry, livestock and other animals that the addition of a humate-like material to the feed promotes growth, better health, and decreased mortality rates. Humic acids have negatively charged ionic sites which singly or in combination chelate or attract and hold positively charged ions and molecules. The carbon chains of the organic matter provide an energy source (food) for microbes which increases their number dramatically. The microbes release enzymes which etch metallic ions or fracture molecules from the food the animal eats which is captured by the humic acids and expedited through the digestive system into contacted cells.

There are three known ways humic acids or humates affect ionic molecular uptake. First, a direct interaction between the humate and cell membrane responsible for ionic transfer. Second, humic acids change the membranes passive permeability allowing greater ion contact and transfer to cellular proteins. And finally, indirect effects, caused by humic acids, can affect transport through changes in the metabolic processes regulating uptake. There is also evidence that intake of humate or humic acid bolsters the immune system and decreases the incidence of illness due to pathogens such as salmonella and e coli.

Fulvic Acid

Nature has a way of processing and refining minerals which is called the Fulvic Acid Phenomenon. Organic fulvic acids are created by micro-organisms in the soil, for the purpose of transporting minerals and nutrients from the soil into a plant. From there, complex photo-synthesis reactions produce the components of all the various parts of the plant. Muco-polysacharrides (complex carbohydrate sugars) flow throughout the plant for nourishment. Some is returned to the roots. There, the micro-organisms are nourished and produce Fulvic Acid to complex with minerals and nutrients to restart the cycle again. In plants, fulvic acid stimulates metabolism, provides respiration, increases metabolism of proteins and activity of multiple enzymes, enhances the permeability of cell membranes, cell division and elongation, aids chlorophyll synthesis, drought tolerance, crop yields, buffers soil pH, assists denitrification by microbes, contributes to electrochemical balance as a donor or an acceptor, decomposes silica to release essential mineral nutrients and detoxifies pollutants such as pesticides and herbicides.

Fulvic acids are small and porous molecules that are easily absorbed from the intestine of a mammal and readily chelate and eliminate trace elements in the bloodstream. Whenever minerals come into contact with fulvic acid, in a water medium, they are naturally dissolved into an ionic form. These minerals literally become part of the fulvic acid itself. Once the minerals meld into the fulvic acid complex, they become bioactive, bioavailable, and organic. Thus, when elemental minerals are transformed into an organic state, through a natural chemical process involving fulvic acid and photosynthesis, they are safe to be used by both humans and animals.

Fulvic acid acts as an important natural detoxifier and protective agent. An important aspect of humic substances is related to their adsorptive interaction with environmental chemicals, either before or after they reach concentrations toxic to living organisms. The toxic herbicide known as Paraquat is rapidly detoxified by humic substances (fulvic acid). Fulvic acids have a special function with respect to the demise of organic compounds applied to soil as pesticides. As the most powerful, natural electrolyte known, fulvic acid restores electrical balance to damaged cells, neutralizes toxins and can eliminate food poisoning within minutes. When it encounters free radicals with unpaired positive or negative electrons, it supplies an equal and opposite charge to neutralize the free radical. Fulvic acid acts as a refiner and transporter of organic materials and cell nutrients.

The majority of research and experimentation that has been done on fulvic acid is in relation to plants. Yet human beings have been ingesting fulvic acid complexes regularly for over 60 years in supplemental form, and for thousands of years from natural food and plant sources. Accumulating testimonials continue to show that the beneficial properties related to plant studies and cell hold true in relation to animal and humans as well. Clinical research on animals and humans show that the most prominent diseases and health problems have been dramatically affected in positive ways by supplementation or treatment with fulvic acid and other preparations enhanced or created with fulvic acid.

Researchers have theorized that mineral deficiency subjects us to more diseases, aging, sickness and destruction of our physical well-being than any other factor in personal health. A great many known aliments, around 60, are directly linked to mineral deficiency. Organic fulvic acids are created by micro-organisms in the soil, for the purpose of transporting minerals and nutrients from the soil into the plant. From there, complex photosynthesis reactions produce the components of all the various parts of the plant. Muco-polysacharrides (complex carbohydrate sugars) flow throughout the plant for nourishment. Some are returned to the roots. There, the micro-organisms are nourished and produce fulvic acid to complex with minerals and nutrients to restart the cycle again.

Fulvic acid enhances the availability of nutrients and makes them more readily absorbable. It also allows minerals to regenerate and prolong the residence time of essential nutrients. It prepares minerals to react with cells. It allows minerals to interact with one another, breaking them down into the simplest ionic forms chelated by the fulvic acid electrolyte process. Fulvic acid acts as a refiner and transporter of organic materials and cell nutrients.

Liquid minerals are often referred to as ionic or colloidal minerals. The absorption factor of ionic minerals greatly exceeds traditional tablet supplements. Fulvic acid plays an important role in the production of ionic or colloidal minerals.) Like fulvic acid, marine phytoplankton, and shilajit are a source of complete natural ionic trace minerals and other important nutrients.

To the science of living cells, fulvic acids are vital in bringing substantial amounts of nutrients and minerals into water solution and delivering their living energies to the living cells. Fulvic acid assists every stage of cellular metabolism and is the most powerful, natural electrolyte known. It restores electrical balance to damaged cells, neutralizes toxins and can eliminate food poisoning within minutes. When it encounters free radicals with unpaired positive or negative electrons, it supplies an equal and opposite charge to neutralize the free radical.

Combinations

Humate and Fulvate have been in use as plant feed for many years. Alexander, U.S. Pat. No. 5,026,416, teaches a method of improving agricultural and horticultural crop yields utilizing a mixture comprising a water-soluble salt of humic acid and either calcium phosphate or ascorbic acid.

The addition of a liquid mixture comprising calcium phosphate or ascorbic acid and a water-soluble salt or humic acid to the growth medium or seed germination medium or agricultural or horticultural crops has demonstrated improved crop yields both in the size of the individual fruit and in the number of the fruit per plant.

U.S. Pat. No. 5,411,569, to Hjersted teaches a particular iron humate blend is taught for commercial application for agricultural crops and turf grasses to prevent or correct for iron deficiency otherwise known as plant chlorosis. Chlorosis can be physically detected by yellowing of leaves in trees, shrubs, and vegetables and yellowing of blades within turf grasses. Chlorosis hinders plant growth or yields and can also lessen food quality.

Humic substances have been shown to bind Antimony(III), aresenite and arsenate. The Chemical modeling of Sb(III)-humics binding of Antimony (III) at different pH values is consistent with two binding sites involving (I) a phenolic entity forming a neutral complex and (ii) a carboxylic entity forming a negatively charged complex. Under environmentally relevant conditions, over 30% of total Sb(III) may be bound to natural organic matter. Buschmann J, and Sigg, L 38(17) Environ Sci Technol 4535-41 (2004). Maximum binding of arsenic was observed at about pH 7, which is consistent with H+ competition for binding sites at low pH values and OH— competition for the arsenic center at high pH. For both oxidation states, conditional distributional coefficients (Dom) values increased with decreasing As/DOC ratios. Dom values were fitted as a function of the As/DOC ratio for As (III) and As(V). Compared to the aquatic humic acid, the terrestrial humic acid had a higher affinity for arsenic binding with 1.5-3 times higher Dom values under the same conditions. Al3+ in excess to arsenic successfully competed for strong binding sites at low As/DOC ratios. Buschmann et al, 40(19) Environ Sci Technol 6015-20 (2006).

Mercury(II) is also bound by aquatic humic substances most optimally at pH 7. Haitzer M, Aiken G R, Ryan J N, 37(11) Environ Sci Technol 2436-41 (2003). The binding of Cu and Pb to humic and fulvic acid was studied in 0.01 M NaNO3 and determined over wide ranges in proton and metal ion activities using three different methods: ligand exchange-adsorptive differential pulse cathodic stripping voltammetry at low humic or fulvic acid concentrations (1-3 mg/L), differential pulse anodic stripping voltammetry at intermediate humic or fulvic acid concentrations (10-20 mg/L), and ion-selective electrodes at high humic or fulvic acid concentrations (approximately 1000 mg/L). The results demonstrate that binding isotherms for Cu and Pb can be measured at low humic or fulvic acid concentration using suitable voltammetric techniques. The binding isotherms for Cu and Pb to humic and fulvic acid obtained at constant pH values in the range of pH 4-8 are shown to be independent of humic and fulvic acid concentration. Christl I, 39(14) Environ Sci Technol 5319-26(2005). Despite clear differences in chemical composition and protonation behavior, the fulvic acid and all humic acid fractions exhibited very similar metal binding behavior. Binding of Cu(II) and Pb(II) generally increased with increasing pH and total metal concentration. At low to moderate metal ion concentrations, Cu(II) was bound more strongly to the humic substances than Pb(II). Only at high free metal concentrations, the amounts of metal ions sorbed were higher for Pb(II) than for Cu(II). Christl I, et al 35(12) Environ Sci Technol 2512-7 (2001).

Humic and Fulvic acid are disclosed as poultry feed in U.S. Patent Appl No. 20080032021, to Faltys. Faltys does not disclose the use of these acids in mammals nor their use in chelating and absorbing trace elements. U.S. Patent App. No. 20030003203, to Williams, discloses an animal feed comprising humic and fulvic acids, for the purposes of minimizing offensive fecal odor that may originate from volatile nitrogen compounds and stimulate growth in animals.

U.S. Patent Appl No., 20080044548, to Hale, describes a method to reduce ammonia emissions in animals, by using a combination of zeolite, humic acid, and fulvic acid as an indigestible cation exchanger combined with an acidogenic substance that produces ammonia cations that can be bound to the ammonia cation exchanger to reduce ammonia emissions in ‘ruminant bird’ manure. Hale does not teach the use of dihydroquercetin in combination with zeolite, nor does Hale teach a method of absorbing, chelating and elimination trace elements by use of this combination.

Shilajit, a composition of fulvic acid with or without humic acid, a native preparation, is described by Ghosal, U.S. Pat. No. 6,869,612, as a human supplement with an abundance of bioactive components for personal care, pharmaceutical and nutritional use. Ghosal does not disclose the combination of Shilajit with zeolite or dihydroquercetin nor does he disclose the use of the composition to enhance the absorption of trace elements and chelate and eliminate them.

### SUMMARY OF THE INVENTION

This inventor has discovered that a combination of dihydroquercetin and zeolite, with the optional addition of humic acid or fulvic acid or both, acts as a powerful chelator and eliminator of trace elements and enhances their absorption from the intestine into the bloodstream and tissues, solving the long-felt need for effective chelation and elimination of trace elements and the absorption of essential minerals. The absorption, chelation and elimination is part of one ongoing process performed by the composition of the invention. In this invention, natural zeolite that has not been heated into a fibrous or crystalline form, and thus remains non-toxic, is optimal.

Dihydroquercetin and its metabolites are known to prevent oxidation by stabilizing transition metal radicals such as copper and iron, which generate the powerful hydroxyl radical. In this invention, dihydroquercetin works as a chelator of ions of metals like copper and iron and prevents them from participating as catalysts for this type of free radical generation. The metal chelating properties of flavonoids suggest that they may play a role in metal-overload diseases and in all oxidative stress conditions involving a transition metal ion. In a comparison study of the interactions of flavonoids with copper and iron ions, the flavonoids showed a higher reducing capacity for copper ions than iron ions.

In a study with dihydroquercetin, also commonly known as taxifolin, the complex of dihydroquercetin with transition metal ions, Fe2+, Fe3+ and Cu2+, showed higher quenching of superoxide radical than uncomplexed dihydroquercetin. The reason for this phenomenon is that these dihydroquercetin-metal complexes were much less subjected to oxidation than the free flavonoid. This study demonstrates that dihydroquercetin-transition metal complexes have superoxide dismutase activity. Kostyuk, V A, et al 428(2) Arch Biochem Biophys 204-8 (2004).

The combination of dihydroquercetin and zeolite has been shown to reduce the amounts of aluminum, mercury, and tin, in in vitro studies (Table 1), while the binding of copper, arsenic, lead, and cadmium is suggested by studies of fulvic and humic acid.

The chelators, DHQ, zeolite, fulvic acid, humic acid, have strong negative charges that enable them to act as a shuttle system transporting positively charged molecules. In this way, the product can have rapid results with “heartburn,” for instance, zeolite removes positively charged hydrogen ions, then exchanges them for other positively charged particles outside of the stomach. It is speculated that it works similarly with excess calcium in the bloodstream, helping to transport Ca+ ions to the muscle where they are most needed. These shuttle effects help balance pH issues and have positive consequences in the overall wellness of the entire system.

This combination enhances and accentuates the chelating effects of the individual ingredients with an affinity range that encompasses a broad spectrum of toxins and heavy metals. DHQ and zeolite in combination have a stronger affinity for aluminum (AL), mercury (Hg) and tin (Sn), while humic acid and fulvic acid have stronger affinities for copper (Cu), arsenic (As), lead (Pb) and cadmium. Further the blend naturally delivers bioavailable nutrients that minimize the usual depletion risks associated with “chemical-based” chelators.

The DHQ and fulvic acid act as absorption boosters; increasing active penetration and begin the chelation process with the smallest particle sizes by accessing microscopic toxins that have accumulated. As the DHQ and fulvic acid continue to work, large toxin complexes loosen and are sequestered by the zeolite or taken up by the humic molecular chains. In this process, the humic acid releases trace minerals that can immediately replace the metals just removed, helping to maintain appropriate cellular function.

### DETAILED DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In the Summary above, the Description of the Invention, and the Claims and Abstract below, reference may be made to particular features (including method steps) of the invention. It is to be understood that this disclosure includes possible combinations of such particular features. For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

The term “comprises” and grammatical equivalents thereof are used herein to mean that other components, ingredients, steps etc. are optionally present. For example, an article “comprising” (or “which comprises”) components A, B and C can consist of (i.e. contain only) components A, B and C, or can contain not only components A, B and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number or the indefinite article “a” (meaning “one”) is used herein to denote the start of a range beginning with that number (which may be a range having an upper limit or no upper limit, depending on the variable being defined). For example “at least one” or “at least a” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. If, in this disclosure, a range is given as “(a first number) to (a second number)” or “(a first number)-(a second number)”, this means a range whose lower limit is the first number and whose upper limit is the second number. For example, 0-10 mm means a range whose lower limit is 0 mm, and whose upper limit is 10 mm.

The term “or” is used herein as a conjunction used to link alternatives in a series of alternatives. The term “and/or” is used herein as a conjunction meaning that either or both of two options may be valid.

In one embodiment of the invention, a dietary supplement composition comprised of dihydroquercetin and zeolite, or their pharmaceutically acceptable salt, ester, or base is described and may be in a dosage form selected from the group consisting of a tablet, capsule, liquid, liposome, inhalant, sublingual tablet, suppository, oral spray and dermal patch, optionally including a pharmaceutically acceptable carrier. In a second embodiment, the dietary supplement composition is further comprised of fulvic acid and humic acid or their pharmaceutically acceptable bases, esters and salts. Both embodiments of the dietary supplement composition, either of dihydroquercetin and zeolite, or dihydroquercetin, zeolite, humic acid, and fulvic acid, is effective for the absorption, chelation and elimination of trace elements.

The trace elements absorbed, chelated and eliminated by the composition may be metalloids, or metals; if metals, transition metals, poor metals, or essential minerals and may be one or more selected from the group consisting of antimony, aluminum, arsenic, barium, beryllium, cadmium, copper, chromium, lead, mercury, nickel, osmium, thallium, tin, vanadium, cobalt, thorium, uranium, radium, strontium, transuraniums, and polonium.

In a specific embodiment, the dietary supplement composition is formulated as a liquid and the dihydroquercetin is present in a range of about 0.2 mg to about 100 mg per ml ; the zeolite is present in a range of about 0.2 to about 100 mg per ml, the humic acid is present in a range of about 1 mg to about 1000 mg per ml; and the fulvic acid is present in a range of about 1 mg to about 1000 mg per ml. In a more specific embodiment, the dihydroquercetin is present In an amount of about 6 mg per ml to about 60 mg per ml; the zeolite is present in an amount of about 3 mg per ml to about 30 mg per ml; the humic acid is present in an amount of about 5 mg per ml to about 300 mg per ml; and the fulvic acid is present in an amount of about 5 mg per ml to about 300 mg per ml. In a most specific embodiment, the dihydroquercetin is present in an amount of about 18 mg per ml, the zeolite is present in an amount of about 10 mg per ml, the humic acid is present in an amount of about 75 mg per ml, and the fulvic acid is present in an amount of about 25 mg per ml, and said liquid is comprised of water. The composition of may be further comprised of trace minerals present in a range of about 4 mg per ml to about 100 mg per ml.

Also disclosed are methods of absorbing, chelating, and eliminating trace elements in a mammal by the administration of the dietary supplement composition in a dose effective to absorb, chelate, and eliminate trace elements to a mammal in need thereof. In a specific embodiment, the dietary supplement is formulated as a liquid further comprising water and said administration is three times a day, twice a day, once a day, every two days or every three days. In a more specific embodiment, the mammal is a human and the administration is in a dose of between 1 drop and 60 drops three times a day, preferably between 3 drops and 30 drops three times a day and most preferably either 5 drops, 10 drops, or 20 drops three times a day. The administration may be performed as 10 drops per day three times a day for one week, 20 drops a day for two weeks, and 5 drops a day for the remainder of the administration up to the life of the human.

Also disclosed are methods of absorbing, chelating, and eliminating trace elements in a mammal by the administration of a dietary supplement composition of zeolite in a dose effective to absorb, chelate and eliminate trace elements to a mammal in need thereof. Said administration may be further comprised of administration of dihydroquercetin in a dose effective to absorb, chelate and eliminate trace elements when administered in combination with said zeolite; and may be further comprised of administration of fulvic acid and humic acid in doses effective to absorb, chelate, and eliminate trace elements when administered in combination with said zeolite and dihydroquercetin.

This method may be used to absorb, chelate, and eliminate trace elements for the purpose of detoxification, to neutralize polycyclic aromatic hydrocarbons from the dietary intake in said mammal in need thereof; to absorb, chelate, and eliminate arsenic, mercury, lead and antimony III from the dietary content of said mammal in need thereof.

The method may be employed for administration to mammals, other than human, and be performed by the addition of the composition to the feed of said mammal. This may be utilized for the absorption, chelation and elimination of trace elements such as the chelation of arsenic, mercury, lead and antimony III from the dietary content of said mammal in need thereof; to detoxify pesticides and herbicides from crops that comprise the dietary intake said mammal in need thereof. Most preferably, said mammal is a companion animal selected from the group of canine, equine and feline species.

### EXAMPLE 1

In in vitro testing, 25 ml of standardized metal solutions containing copper [Cu], arsenic [As], lead [Pb], cadmium [Cd], aluminum [Al], mercury [Hg], and tin [Tn] was added to a 5 ml solution of zeolite and dihydroquercetin prepared according to this invention, agitated and allowed to set for 5 hours. 86.9% of Tn, 79% of Hg, and 57.8% of Al were removed, and the other elements removed in lesser amounts. See Table 1.

While this invention has been particularly shown and described with references to specific embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method of absorbing, chelating and eliminating trace elements in a human by the administration of the dietary supplement composition of claim 15 in a dose effective to absorb, chelate, and eliminate trace elements to a mammal in need thereof.

2. The method of claim 1 wherein said dietary supplement is formulated as a liquid further comprising water and said administration is three times a day, twice a day, once a day, every two days or every three days.

3. The method of claim 2 where said administration is in a dose of between 1 drop and 60 drops three times a day.

4. The method of claim 3 wherein said dose is between 3 drops and 30 drops three times a day.

5. The method of claim 3 wherein said dose is 5 drops three times a day.

6. The method of claim 3 wherein said dose is 10 drops three times a day.

7. The method of claim 3 wherein said dose is 20 drops three times a day.

8. The method of claim 3 wherein said dose is 10 drops per day three times a day for one week, 20 drops a day for two weeks, and 5 drops a day for the remainder of said administration up to the life of the human.

9. A method of absorbing, chelating, and eliminating trace elements in a mammal by the administration of a dietary supplement composition of dihydroquercetin and zeolite in a dose effective to absorb, chelate, and eliminate trace elements to a mammal in need thereof.

10. The method of claim 9, further comprising the administration of a dietary supplement composition comprised of fulvic acid and humic acid in doses effective to absorb and chelate trace elements when administered in combination with said zeolite and dihydroquercetin.

11. The method of claim 10 wherein said absorbing, chelating and eliminating of trace elements is performed for the purpose of detoxification.

12. The method of claim 11 wherein said absorption, chelation and elimination of trace elements neutralizes polycyclic aromatic hydrocarbons from the dietary intake in said mammal in need thereof.

13. The method of claim 9 wherein said absorption, chelation and elimination of trace elements is of copper, arsenic, lead, cadmium, aluminum, mercury, tin, and antimony III from the dietary content of said mammal in need thereof.

14. The method of claim 9 wherein said absorption, chelation and elimination of trace elements is the chelation of copper.

15. The method of claim 9 wherein said absorption, chelation and elimination of trace elements is the chelation of arsenic.

16. The method of claim 9 wherein said absorption, chelation and elimination of trace elements is the chelation of lead.

17. The method of claim 9 wherein said absorption, chelation and elimination of trace elements is the chelation of cadmium.

18. The method of claim 9 wherein said absorption, chelation and elimination of trace elements is the chelation of aluminum.

19. The method of claim 9 wherein said absorption, chelation and elimination of trace elements is the chelation of mercury.

20. The method of claim 9 wherein said absorption, chelation and elimination of trace elements is the chelation of tin.

21. The method of claim 9 wherein said absorption, chelation and elimination of trace elements is the chelation of antimony II.

22. The method of claim 9 wherein said absorption, chelation and elimination of trace elements detoxifies pesticides and herbicides from crops that comprise the dietary intake said mammal in need thereof.

23. The method of claim 9 wherein said administration is to mammals, other than human, and is performed by the addition of the composition to the feed of said mammal.

24. The method of claim 9 wherein said absorption, chelation and elimination of trace elements is of copper, arsenic, lead, cadmium, aluminum, mercury, tin, and antimony III from the dietary content of said mammal, other than human, in need thereof.

25. The method of claim 9 wherein said absorption, chelation and elimination of trace elements occurs in the intestine of said mammal in need thereof.

26. The method of claim 9 wherein said absorption, chelation and elimination of trace elements occurs in the blood of said mammal in need thereof.

27. The method of claim 9 wherein said absorption, chelation and elimination of trace elements occurs in the organs of said mammal in need thereof.

28. The method of claim 9 wherein said mammal is a companion animal selected from the group of canine, equine and feline species.
